SEQUENCE LISTING (40 sequences)

SEQ ID NO: 1 - protein, 129 aa, synthetic construct
EVQVVESGGDLVQPGGSLKLSCQASGFSLTYYAIGWFRQAPGKEREEISCISGDGGMTYYADPVKGRFTISRDNGRNTVYLQMNSLKPEDTAVYYCAADVYYNGNYYSRSCNPAESKYWGQGTQVTVSA

SEQ ID NO: 2 - protein, 129 aa, synthetic construct
EVQVVESGGGLVQPGGSLRLSCAASGFSLTYYAIGWFRQAPGKEREGVACISGGGDQLYYTDSVKGRFTISRDNAKNTVYLHMNSLKPEDTAVYYCAADVYYSGSYNYRSCNPAESKYWGQGTQVTVSS

SEQ ID NO: 3 - protein, 129 aa, synthetic construct
QVQLVESGGGLVQPGGSLRLSCAASGFSLEYYAIGWFRQTPGKEREAISCIDSPAKSTYYADSVKGRFTISRDNSKNTVYLQMNSLKAEDTAVYYCAADVYYRGSYLYRSCNPTESKYWGQGTQVTVSS

SEQ ID NO: 4 - protein, 129 aa, synthetic construct
QVQLVESGGGLVQPGGSLRLSCAASGFSLEYYAIGWFRHIPGKEREAVSCISSPGDSTWYVDSVKGRFTISRDNAKNTVYLQMNSLKPEDTAVYHCAADVYYSGSYLSRSCNPHESKYWGQGTQVTVSS

SEQ ID NO: 5 - protein, 25 aa, synthetic construct
EVQVVESGGDLVQPGGSLKLSCQAS

SEQ ID NO: 6 - protein, 25 aa, synthetic construct
EVQVVESGGGLVQPGGSLRLSCAAS

SEQ ID NO: 7 - protein, 25 aa, synthetic construct
QVQLVESGGGLVQPGGSLRLSCAAS

SEQ ID NO: 8 - protein, 8 aa, synthetic construct
GFSLTYYA

SEQ ID NO: 9 - protein, 8 aa, synthetic construct
GFSLEYYA

SEQ ID NO: 10 - protein, 17 aa, synthetic construct
IGWFRQAPGKEREEISC

SEQ ID NO: 11 - protein, 17 aa, synthetic construct
IGWFRQAPGKEREGVAC

SEQ ID NO: 12 - protein, 17 aa, synthetic construct
IGWFRQTPGKEREAISC

SEQ ID NO: 13 - protein, 17 aa, synthetic construct
IGWFRHIPGKEREAVSC

SEQ ID NO: 14 - protein, 8 aa, synthetic construct
ISGDGGMT

SEQ ID NO: 15 - protein, 8 aa, synthetic construct
ISGGGDQL

SEQ ID NO: 16 - protein, 8 aa, synthetic construct
IDSPAKST

SEQ ID NO: 17 - protein, 8 aa, synthetic construct
ISSPGDST

SEQ ID NO: 18 - protein, 38 aa, synthetic construct
YYADPVKGRFTISRDNGRNTVYLQMNSLKPEDTAVYYC

SEQ ID NO: 19 - protein, 38 aa, synthetic construct
YYTDSVKGRFTISRDNAKNTVYLHMNSLKPEDTAVYYC

SEQ ID NO: 20 - protein, 38 aa, synthetic construct
YYADSVKGRFTISRDNSKNTVYLQMNSLKAEDTAVYYC

SEQ ID NO: 21 - protein, 38 aa, synthetic construct
WYVDSVKGRFTISRDNAKNTVYLQMNSLKPEDTAVYHC

SEQ ID NO: 22 - protein, 22 aa, synthetic construct
AADVYYNGNYYSRSCNPAESKY

SEQ ID NO: 23 - protein, 22 aa, synthetic construct
AADVYYSGSYNYRSCNPAESKY

SEQ ID NO: 24 - protein, 22 aa, synthetic construct
AADVYYRGSYLYRSCNPTESKY

SEQ ID NO: 25 - protein, 22 aa, synthetic construct
AADVYYSGSYLSRSCNPHESKY

SEQ ID NO: 26 - protein, 11 aa, synthetic construct
WGQGTQVTVSA

SEQ ID NO: 27 - protein, 11 aa, synthetic construct
WGQGTQVTVSS

SEQ ID NO: 28 - DNA, 387 bp, synthetic construct
  source 1..387: mol_type = other DNA
gaggtgcaggtggtggagtctgggggagacttggtgcagcctggggggtctctgaaactctcctgtcaagcctctggattcagtttgacgtattatgccataggctggttccgccaggccccaggaaaggaacgtgaggaaatctcatgtattagtggtgatggtggtatgacatactatgcagaccccgtgaagggccgattcaccatctccagagacaacggcaggaacacggtgtatctgcaaatgaacagcctgaaacctgaggacacagccgtttattactgtgcagcagacgtatactataatggcaattactacagccgctcatgtaaccctgctgagtcaaagtactggggccaggggacccaggtcaccgtctccgca

SEQ ID NO: 29 - DNA, 387 bp, synthetic construct
  source 1..387: mol_type = other DNA
gaggtgcaggtggtggagtctgggggaggcttggtgcaacctggggggtctctgagactctcctgtgcagcctctggattcagtttgacttattatgccataggctggttccgccaggccccagggaaggagcgtgagggggttgcatgtattagtggtggcggtgaccagctttattatacagactccgtgaagggccgattcaccatctccagagacaatgccaagaacacggtgtatctgcacatgaacagcctgaaacctgaggacacagccgtttattactgtgcagcagatgtgtactatagtggtagttacaactaccggtcatgtaaccctgctgagtcaaaatactggggccaggggacccaggtcaccgtctcctca

SEQ ID NO: 30 - DNA, 387 bp, synthetic construct
  source 1..387: mol_type = other DNA
caggtgcagctcgtggagtccgggggaggcttggtgcagcctggggggtctctgagactctcctgtgcagcctctggattcagtttggagtattatgccataggctggttccgccagaccccaggaaaggagcgtgaggcgatctcatgtattgatagtcctgctaagagcacgtactatgcagactccgtgaagggccgattcaccatctccagagacaactccaagaacacggtgtatctgcaaatgaacagcctgaaagctgaggacacagccgtttattactgtgcagcagatgtatactatcgcggtagttacttgtaccgctcatgtaaccctactgagtcaaaatactggggccaggggacccaggtcaccgtctcctca

SEQ ID NO: 31 - DNA, 387 bp, synthetic construct
  source 1..387: mol_type = other DNA
caggtgcagctcgtggagtcggggggaggcttggtgcagcctggggggtctctgagactctcctgtgcagcctctggattcagtttggagtattatgccataggctggttccgccacatcccagggaaagagcgtgaggcggtctcatgtattagtagtcctggtgatagtacgtggtatgtagactccgtgaagggccgattcaccatctccagagacaacgccaagaacacggtgtatctgcaaatgaacagcctgaaacctgaggacacagccgtttatcactgtgcagcagacgtatactatagtggtagttacctgagccgctcatgtaacccgcatgaatcaaaatactggggccaggggacccaggtcaccgtctcctca

SEQ ID NO: 32 - DNA, 969 bp, synthetic construct
  source 1..969: mol_type = other DNA
atgggctggtcctgcatcatcctgtttctggtcgctaccgccacaggcgtccactcctccctgaggggcagagacgcccccgctccaacaccttgcgtgcctgccgagtgcttcgatctgctggtgaggcactgcgtggcctgcggcctgctgaggacccctaggcctaagcccgccggcgctagcagcccagctcctagaacagccctgcagccccaggagagcgtgggcgctggagctggagaggccgctctgcccctgcctggcctggaaccaaaatcttgtgacaaaacccacacatgcccaccttgtcccgcccctgaactgctgggcggaccttctgtctttctgttcccccccaaacccaaggatacactgatgatctctagaacccccgaggtcacatgtgtcgtcgtggatgtgtcccatgaggaccctgaagtgaaattcaactggtacgtggacggagtggaagtccataacgccaaaaccaaaccacgagaggaacagtacaatagcacatatcgagtcgtgtctgtgctgactgtgctgcatcaggattggctgaacggcaaggaatacaaatgtaaagtgtccaataaggcactgcccgctcctattgaaaaaacaatttcaaaggcaaaaggccagcctcgtgaacctcaggtgtacacactgccaccttctcgggaggaaatgaccaaaaaccaggtgtcactgacctgtctggtcaagggcttttacccttccgatattgctgtcgagtgggagagtaacggccagcccgaaaacaactacaaaaccacccctcctgtgctggattccgatggctcattcttcctgtactctaaactgaccgtggataagagtcgctggcagcagggcaatgtgttttcttgctccgtgatgcatgaggcactgcacaaccactacacccagaaatccctgtcactgtctcccggaaaataa

SEQ ID NO: 33 - protein, 322 aa, synthetic construct
MGWSCIILFLVATATGVHSSLRGRDAPAPTPCVPAECFDLLVRHCVACGLLRTPRPKPAGASSPAPRTALQPQESVGAGAGEAALPLPGLEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - DNA, 18 bp, synthetic construct
  source 1..18: mol_type = other DNA
cttggtggtcctggctgc

SEQ ID NO: 35 - DNA, 23 bp, synthetic construct
  source 1..23: mol_type = other DNA
ggtacgtgctgttgaactgttcc

SEQ ID NO: 36 - DNA, 47 bp, synthetic construct
  source 1..47: mol_type = other DNA
catgccatgactgtggcccaggcggcccagktgcagctcgtggagtc

SEQ ID NO: 37 - DNA, 47 bp, synthetic construct; identical to SEQ ID NO: 36
  source 1..47: mol_type = other DNA

SEQ ID NO: 38 - DNA, 51 bp, synthetic construct
  source 1..51: mol_type = other DNA
catgccatgactcgcggccggcctggccgctggggtcttcgctgtggtgcg

SEQ ID NO: 39 - DNA, 555 bp, synthetic construct
  source 1..555: mol_type = other DNA
atgaggcgagggccccggagcctgcggggcagggacgcgccagcccccacgccctgcgtcccggccgagtgcttcgacctgctggtccgccactgcgtggcctgcgggctcctgcgcacgccgcggccgaaaccggccggggccagcagccctgcgcccaggacggcgctgcagccgcaggagtcggtgggcgcgggggccggcgaggcggcgctgcccctgcccgggctgctctttggcgcccccgcgctgctgggcctggcactggtcctggcgctggtcctggtgggtctggtgagctggaggcggcgacagcggcggcttcgcggcgcgtcctccgcagaggcccccgacggagacaaggacgccccagagcccctggacaaggtcatcattctgtctccgggaatctctgatgccacagctcctgcctggcctcctcctggggaagacccaggaaccaccccacctggccacagtgtccctgtgccagccacagagctgggctccactgaactggtgaccaccaagacggccggccctgagcaacaatag

SEQ ID NO: 40 - protein, 184 aa, synthetic construct
MRRGPRSLRGRDAPAPTPCVPAECFDLLVRHCVACGLLRTPRPKPAGASSPAPRTALQPQESVGAGAGEAALPLPGLLFGAPALLGLALVLALVLVGLVSWRRRQRRLRGASSAEAPDGDKDAPEPLDKVIILSPGISDATAPAWPPPGEDPGTTPPGHSVPVPATELGSTELVTTKTAGPEQQ

(19) United States
(12) Patent Application Publication
(10) Pub. No.: US 2026/0209373 A1
(43) Pub. Date: Jul. 23, 2026

(54) ANTIGEN BINDING POLYPEPTIDE AGAINST HUMAN B LYMPHOCYTE STIMULATOR FACTOR RECEPTOR BAFFR AND USE THEREOF

(71) Applicant: Tianjin Medical University General Hospital, Tianjin (CN)

(72) Inventors: Qiang LIU, Tianjin (CN); Guoqiang Chang, Tianjin (CN)

(21) Appl. No.: 19/454,735

(22) Filed: Jan. 21, 2026

Related U.S. Application Data
(63) Continuation of application No. PCT/CN2024/089314, filed on Apr. 23, 2024.

(30) Foreign Application Priority Data
Sep. 26, 2023 (CN) 202311250418.3

(51) Int. Cl. C07K 16/28

(52) U.S. Cl. CPC C07K 16/2878; C07K 2317/569; C07K 2319/00

(57) ABSTRACT

The present invention relates to an antigen binding polypeptide against human B lymphocyte stimulator factor receptor BAFFR and use thereof, and particularly to an anti-BAFFR nanobody. The present invention further provides a nucleotide sequence encoding the antigen binding polypeptide, an expression vector or a host cell comprising the nucleotide sequence, and a method for producing the nanobody. The present invention further provides a composition comprising the nanobody of the present invention and use thereof.

### CROSS-REFERENCE TO RELATED APPLICATIONS

This application is a continuation of International Application No. PCT/CN2024/089314, filed on Apr. 23, 2024, which claims priority to Chinese Patent Application No. 202311250418.3, filed on Sep. 26, 2023. The disclosures of the aforementioned applications are hereby incorporated by reference in their entireties.

### STATEMENT OF SEQUENCE LISTING

The Sequence Listing XML, which is part of this application, is incorporated herein by reference in its entirety. The Sequence Listing XML is submitted as a file named PR34294WHUS_Sequence_listing.xml, created on Oct. 21, 2025, and having a size of 39936 bytes.

### TECHNICAL FIELD

The present invention relates to the technical field of molecular biology and biomedical science, and particularly to a nanobody (VHH antibody) specifically binding to B lymphocyte stimulator factor receptor BAFFR, and a preparation method and related use thereof.

### BACKGROUND

B lymphocyte stimulator factor (BAFF, also referred to as TNFSF13B) is a cytokine belonging to the tumor necrosis factor family, a key factor for the survival and maturation of B cells. BAFF can promote the survival of B cells by promoting their proliferation and/or differentiation, and plays an important role in the production and maintenance of mature B cells. B cell development disorder may lead to immune deficiency, escape of autoimmune B cells and production of autoimmune antibodies, and is a key factor causing the occurrence and continuous development of autoimmune diseases and B cell-related tumors. As an important immunomodulatory molecule, BAFF has strong chemotaxis to B cells, and plays a key role in maintaining the dynamic balance of B cells. Abnormal high expression of BAFF can inhibit the death of autoreactive B cells and induce the production of autoimmune antibodies, thus breaking the homeostasis of immune tolerance and leading to the occurrence of autoimmune diseases or tumors.

Studies show that BAFF is involved in the progress of many autoimmune diseases, and has an obviously increased expression level in many autoimmune diseases. For example, in patients with systemic lupus erythematosus, BAFF is constantly highly expressed, and the concentration of BAFF in the patients' serum is positively correlated with the titer of anti-dsDNA antibodies. In addition, the expression level of BAFF in the sera of patients with Sjogren's syndrome, rheumatoid arthritis and multiple sclerosis is also increased significantly is also increased significantly.

BAFF can bind to three membrane receptors of the TNFR family on the surface of B cells, The three membrane receptors are respectively B lymphocyte stimulator factor receptor (BAFF receptor, BAFFR), transmembrane activator and calcium modulator and cyclophilin ligand interactor (TACI), and B cell maturation antigen (BCMA). The binding of BAFF and BAFFR plays the most important role in the maturation of peripheral B cells.

At present, B cell depletion by CD19 or CD20 antibody is the main treatment for autoimmune diseases, but the treatment effect is undesirable. In one aspect, the CD19 or CD20 antibody will target all naive and mature B cells, which will easily lead to immune deficiency and even infection in patients. In another aspect, the CD19 or CD20 antibody may reduce the patients' response to vaccines, such as COVID-19 vaccine. In addition, autoantibodies exist in patients with autoimmune diseases, such as dsDNA antibody in patients with systemic lupus erythematosus, MOG antibody in patients with multiple sclerosis, and AQP4 antibody in patients with neuromyelitis optica. CD20 monoclonal antibody can not reduce the concentration of these pathogenic autoantibodies (30-70%) in the patients.

At present, therapeutic antibodies targeting BAFFR have been developed, such as Lanalumab. Lanalumab is a human-mouse chimeric antibody. Generally, such antibodies are produced by obtaining a gene encoding a variable region of a target antibody from hybridoma cells that produce a mouse monoclonal antibody, then recombining the gene with a human constant region gene, cloning the recombined gene into a suitable vector, and then transferring the vector into a recipient cell for expression. Therefore, such antibodies has a low humanization rate of generally 60-70%, and potentially has strong immunogenicity and low safety.

In view of the limitations and complexity of the current treatment methods, new treatment methods and/or products are still needed in the art, which can eliminate specific pathogenic B cell subsets or regulate the activation and function of B cells in a more precise way, to treat B cells related diseases, such as tumors or autoimmune diseases.

### SUMMARY

To solve the above problems, through the phage display technology in combination with a high-throughput expression platform, a nanobody targeting BAFFR is screened out. The nanobody has the advantages such as simple humanization, high affinity, high stability, ability to express in microorganisms, low immunogenicity, high solubility, powerful permeability, and ability to recognize hidden epitopes. Accordingly, the present invention is accomplished. In addition, the present invention further provides a method for producing the nanobody, and use of the nanobody in the treatment or diagnosis of autoimmune diseases and tumors.

Therefore, in a first aspect, the present invention provides an antigen binding polypeptide specific for human BAFFR. The antigen binding polypeptide comprises a complementarity determining region 1 (CDR1), a complementarity determining region 2 (CDR2) and a complementarity determining region 3 (CDR3) selected from:
the CDR1 having an amino acid sequence as shown in SEQ ID NO: 8 or 9,
the CDR2 having an amino acid sequence as shown in any one of SEQ ID NOs: 14-17, and
the CDR3 having an amino acid sequence as shown in any one of SEQ ID NOs: 22-25.

In a specific embodiment, the CDR1 and CDR2 and CDR3 are selected from the groups consisting of
(a) the CDR1 having an amino acid sequence as shown in SEQ ID NO: 8;
the CDR2 having an amino acid sequence as shown in SEQ ID NO: 14; and
the CDR3 having an amino acid sequence as shown in SEQ ID NO: 22;
(b) the CDR1 having an amino acid sequence as shown in SEQ ID NO: 8;
the CDR2 having an amino acid sequence as shown in SEQ ID NO: 15; and
the CDR3 having an amino acid sequence as shown in SEQ ID NO: 23;
(c) the CDR1 having an amino acid sequence as shown in SEQ ID NO: 9;
the CDR2 having an amino acid sequence as shown in SEQ ID NO: 16; and
the CDR3 having an amino acid sequence as shown in SEQ ID NO: 24; or
(d) the CDR1 having an amino acid sequence as shown in SEQ ID NO: 9;
the CDR2 having an amino acid sequence as shown in SEQ ID NO: 17; and
the CDR3 having an amino acid sequence as shown in SEQ ID NO: 25.

Preferably, the antigen binding polypeptide is an anti-BAFFR antibody. Preferably, the antibody comprises FR1, CDR1, FR2, CDR2, FR3, CDR3, and FR4, for example, as a heavy chain variable region.

Further preferably the anti-BAFFR antibody is anti-BAFFR nanobody, comprising FR1, CDR1, FR2, CDR2, FR3, CDR3, and FR4.

Further preferably, FR1 has an amino acid sequence selected from the sequence as shown in any one of SEQ ID NOs: 5-7, FR2 has an amino acid sequence selected from the sequence as shown in any one of SEQ ID NOs: 10-13, FR3 has an amino acid sequence selected from the sequence as shown in any one of SEQ ID NOs: 18-21, and/or FR4 has an amino acid sequence selected from the sequence as shown in any one of SEQ ID NOs: 26-27.

More specifically, the anti-BAFFR nanobody has an amino acid sequence selected from any one of
(a) an amino acid sequence as shown in SEQ ID NO: 1 (nanobody NB467-55);
(b) an amino acid sequence as shown in SEQ ID NO: 2 (nanobody NB467-46);
(c) an amino acid sequence as shown in SEQ ID NO: 3 (nanobody NB467-35); and
(d) an amino acid sequence as shown in SEQ ID NO: 4 (nanobody NB467-8).

In a second aspect, the present invention further provides an isolated nucleic acid molecule, comprising a nucleotide sequence encoding the antigen binding polypeptide. Preferably, the nucleic acid molecule comprises or consists of a nucleotide sequence as shown in any one of SEQ ID NOs: 28-31.

In a third aspect, the present invention further provides a recombinant expression vector, comprising the isolated nucleic acid molecule according to the second aspect.

In a fourth aspect, the present invention further provides a host cell, comprising the nucleic acid molecule according to the second aspect or the recombinant expression vector according to the third aspect. Preferably, the host cell is a eukaryotic cell, further preferably, mammalian cells.

In a fifth aspect, the present invention further provides a pharmaceutical composition, comprising the antigen binding polypeptide according to the first aspect and a pharmaceutically acceptable carrier.

In a sixth aspect, the present invention further provides a fusion protein comprising the antigen binding polypeptide according to the first aspect. For example, the fusion protein is a chimeric antigen receptor, where the chimeric antigen receptor has an extracellular domain comprising the antigen binding polypeptide according to the first aspect, and the chimeric antigen receptor further comprises transmembrane domain and an intracellular domain.

In a seventh aspect, the present invention further provides a method for producing an anti-human BAFFR nanobody, which comprises expressing the isolated nucleic acid molecule according to the second aspect, expressing the recombinant vector according to the third aspect, or culturing the host cell according to the fourth aspect, and optionally purifying.

In an eighth aspect, the present invention further provides use of the antigen binding polypeptide according to the first aspect, the isolated nucleic acid molecule according to the second aspect, the recombinant expression vector according to the third aspect or the host cell according to the fourth aspect in the preparation of drugs for treating or preventing autoimmune diseases or tumors.

Preferably, the autoimmune disease is selected from the group consisting immune diseases of the nervous system, such as multiple sclerosis, neuromyelitis optica, and myasthenia gravis, and other autoimmune diseases such as rheumatoid arthritis, systemic lupus erythematosus, and Sjogren's syndrome.

Preferably, the tumors include hematomas, such as B-cell lymphomas, such as Hodgkin's lymphoma and nodular lymphocyte-predominant Hodgkin lymphoma, and non-Hodgkin's lymphomas, such as diffuse large B-cell lymphoma, follicular lymphoma, mucosa-associated lymphoid tissue lymphoma (MALT), small lymphocytic lymphoma/chronic lymphocytic leukemia, and, mantle cell lymphoma (MCL).

In a ninth aspect, the present invention further provides a method for treating or preventing autoimmune diseases or tumors in a subject, comprising administering to the subject a therapeutically effective amount of the antigen binding polypeptide according to the first aspect, the isolated nucleic acid molecule according to the second aspect, the recombinant expression vector according to the third aspect, the host cell according to the fourth aspect or the pharmaceutical composition according to the fifth aspect.

The nanobody of the present invention has, in addition to the excellent performances of nanobodies such as low molecular weight and high humanization level, excellent antigen binding specificity, ability to competitively block BAFF-BAFFR interaction, and ADCC activity, Thus, the nanobody has at least the following advantages:
(1) The anti-human BAFFR nanobody of the present invention has high affinity to human BAFFR, has stronger ADCC phagocytosis compared with the existing anti-human BAFFR antibody, and can induce macrophages to phagocytize tumor cells, with an EC₅₀ lower than that of a reference antibody.
(2) The anti-human BAFFR nanobody of the present invention has stronger ability to competitively block BAFF-BAFFR interaction.
(3) Compared with monoclonal antibodies, especially chimeric antibodies, the anti-human BAFFR nanobody of the present invention has high safety and low immunogenicity.
(4) The nanobody provided in the present invention has a small molecular weight, so it has strong tissue permeability, and can directly penetrate some internal barriers, thus facilitating the diagnosis and targeted treatment of diseases.

### BRIEF DESCRIPTION OF THE DRAWINGS

FIG. 1 shows the affinity of a nanobody and a control antibody to 293T cells overexpressing BAFFR on the surface detected by flow cytometry, in which MFI refers to the median fluorescence intensity.

FIG. 2 shows the median inhibition concentration (IC50 value) of a nanobody and a control antibody on BAFF-BAFFR interaction determined by ELISA.

FIG. 3 shows the effective concentration (EC50 value) of a nanobody and a control antibody to induce the cytotoxicity of T cells on 293T cells overexpressing BAFFR determined by ELISA.

### DETAILED DESCRIPTION

### I. Terms

In the context of the present invention, unless otherwise indicated, BAFFR is human BAFFR (Gene ID: 115650. Nucleotide: NM_052945.4, and Amino acid: NP_443177.1), having an amino acid sequence and a nucleotide sequence respectively as shown in SEQ ID NO: 39 and SEQ ID NO: 40.

“Antigen binding polypeptide” refers to a polypeptide sequence that specifically binds to an antigen, and may be a modified or unmodified intact protein, polypeptide, polypeptide, or an antigen binding fragment with an immunological function.

“Binding BAFFR” or “binding to BAFFR” refers to an interaction that allows for specific binding to BAFFR.

“Nanobody”, also known as VHH antibody, refers to a single domain antibody obtained from camelidae and consisting exclusively of a single variable domain on the heavy chain. The nanobody or antigen binding fragment thereof can be produced, for example, by a recombinant technique, a phage display technique, a synthetic technique such as CDR grafting, or a combination of these or other techniques known in the art.

“Affinity” refers to the total strength of all non-covalent interactions between a single binding site of a molecule (such as an antibody) and its binding partner (such as an antigen). Unless otherwise indicated, herein, the “binding affinity” refers to the inherent binding affinity that reflects the 1:1 interaction between an antibody and an antigen. The affinity can be measured by common methods known in the art, including those known in the art and described herein.

“Competition”, when used with reference to an antigen binding polypeptide that competes for the same epitope (for example, a neutralizing antigen binding polypeptide or a neutralizing antibody), means the competition between antigen binding polypeptides. The competition between antigen binding polypeptides can be determined by an assay in which the antigen binding polypeptide to be detected (e.g., an antibody or an immunologically functional fragment thereof) prevents or inhibits (e.g., reduces) the specific binding of a reference antigen binding polypeptide (e.g., a ligand or a reference antibody) to a common antigen (e.g., BAFFR or a fragment thereof). Many types of competitive binding assays can be used to determine whether one antigen binding polypeptide competes with another. The inhibition of competitive binding can be measured by measuring the amount of a label that bind to a solid surface or cells in the presence of the detected antigen binding polypeptide. Usually, the detected antigen binding polypeptide is in excess.

“Treatment” means administering a therapeutic agent for internal or topical use, such as a composition containing an antigen binding polypeptide that specifically binds to BAFFR, to a patient who has one or more disease symptoms. Generally, the therapeutic agent is administered to a patient or population to be treated in an amount effective to relieve one or more disease symptoms by inducing the regression of such symptoms or inhibiting the development of such symptoms to any clinically measurable extent.

The “effective amount” includes an amount sufficient to improve or prevent symptoms or signs of a medical disorder. The effective amount also means an amount sufficient to allow or promote diagnosis. The effective amount for a specific patient or animal subject may vary depending on such factors as the disorder to be treated, the general health of the patient, the method, route and dosage of administration, and the severity of side effects. The effective amount can be the maximum dose or dosage regimen to avoid significant side effects or toxic effects.

The “pharmaceutical composition” means a mixture containing one or more antigen binding polypeptides described herein and other pharmaceutical components, such as physiologically/pharmaceutically acceptable carriers and excipients. The pharmaceutical composition serves to promote the administration to an organism, to facilitate the absorption of the active ingredients to exert a biological activity.

### II. Antigen Binding Polypeptide and Nanobody

The present invention provides antigen binding polypeptides specifically binding BAFFR. In some embodiments, the antigen binding polypeptide specifically binding BAFFR is an antibody, preferably a nanobody.

In some embodiments, the antibody, preferably nanobody, comprises a “complementarity determining region 1 (CDR1)”, a complementarity determining region 2 (CDR2) and a complementarity determining region 3 (CDR3).

In a preferred embodiment, the CDR1 comprises or consists of an amino acid sequence as shown in SEQ ID NO: 8 or 9, or comprises or consists of an amino acid sequence that differs from the amino acid sequence as shown in SEQ ID NO: 8 or 9 by 1 or 2 amino acids. In a further preferred embodiment, the CDR1 has an amino acid sequence as shown in SEQ ID NO: 8 or SEQ ID NO: 9.

In a preferred embodiment, the CDR2 comprises or consists of an amino acid sequence as shown in any one of SEQ ID NO: 14-17, or comprises or consists of an amino acid sequence that differs from the amino acid sequence as shown in any one of 14-17 by 1 or 2 amino acids. In a further preferred embodiment, the CDR2 has an amino acid sequence as shown in any one of 14-17.

In a preferred embodiment, the CDR3 comprises or consists of an amino acid sequence as shown in any one of SEQ ID NO: 22-25, or comprises or consists of an amino acid sequence that differs from the amino acid sequence as shown in any one of 22-25 by 1 or 2 amino acids. In a further preferred embodiment, the CDR2 has an amino acid sequence as shown in any one of 22-25.

In a further preferred embodiment, the CDR1, CDR2 and CDR3 are selected from the groups consisting of
(a) the CDR1 having an amino acid sequence as shown in SEQ ID NO: 8;
the CDR2 has an amino acid sequence as shown in SEQ ID NO: 14; and
the CDR3 has an amino acid sequence as shown in SEQ ID NO: 22;
(b) the CDR1 having an amino acid sequence as shown in SEQ ID NO: 8;
the CDR2 has an amino acid sequence as shown in SEQ ID NO: 15; and
the CDR3 has an amino acid sequence as shown in SEQ ID NO: 23;
(c) the CDR1 having an amino acid sequence as shown in SEQ ID NO: 9;
the CDR2 has an amino acid sequence as shown in SEQ ID NO: 16; and
the CDR3 has an amino acid sequence as shown in SEQ ID NO: 24; or
(d) the CDR1 having an amino acid sequence as shown in SEQ ID NO: 9;
the CDR2 has an amino acid sequence as shown in SEQ ID NO: 17; and
the CDR3 has an amino acid sequence as shown in SEQ ID NO: 25.

In a specific embodiment, the antibody, preferably nanobody, comprises four framework regions (FR) arranged alternately with the three CDRs. Specifically, the antibody, preferably nanobody, comprises FR1, CDR1, FR2, CDR2, FR3, CDR3, and FR4 in sequence.

In a specific embodiment, the antibody, preferably nanobody, comprises or consists of an amino acid sequence shown below:
(a) an amino acid sequence as shown in SEQ ID NO: 1, or an amino acid sequence having at least 95%, at least 96%, at least 97%, at least 98%, or at least 99% sequence identity to the amino acid sequence;
(b) an amino acid sequence as shown in SEQ ID NO: 2, or an amino acid sequence having at least 95%, at least 96%, at least 97%, at least 98%, or at least 99% sequence identity to the amino acid sequence;
(c) an amino acid sequence as shown in SEQ ID NO: 3, or an amino acid sequence having at least 95%, at least 96%, at least 97%, at least 98%, or at least 99% sequence identity to the amino acid sequence; and
(d) an amino acid sequence as shown in SEQ ID NO: 4, or an amino acid sequence having at least 95%, at least 96%, at least 97%, at least 98%, or at least 99% sequence identity to the amino acid sequence.

The present invention further provides a nucleotide sequence encoding the antigen binding polypeptide. In a preferred embodiment, the nucleotide sequence is selected from the group consisting of,
(a) a nucleotide sequence as shown in SEQ ID NO: 28, or a nucleotide sequence having at least 95%, at least 96%, at least 97%, at least 98%, or at least 99% sequence identity to the nucleotide sequence and encoding the amino acid sequence as shown in SEQ ID NO: 1;
(b) a nucleotide sequence as shown in SEQ ID NO: 29, or a nucleotide sequence having at least 95%, at least 96%, at least 97%, at least 98%, or at least 99% sequence identity to the nucleotide sequence and encoding the amino acid sequence as shown in SEQ ID NO: 2;
(c) a nucleotide sequence as shown in SEQ ID NO: 30, or a nucleotide sequence having at least 95%, at least 96%, at least 97%, at least 98%, or at least 99% sequence identity to the nucleotide sequence and encoding the amino acid sequence as shown in SEQ ID NO: 3; and
(d) a nucleotide sequence as shown in SEQ ID NO: 31, or a nucleotide sequence having at least 95%, at least 96%, at least 97%, at least 98%, or at least 99% sequence identity to the nucleotide sequence and encoding the amino acid sequence as shown in SEQ ID NO: 4.

The present invention further provides an expression vector comprising a nucleotide molecule encoding the antigen binding polypeptide of the present invention. The expression vector may contain a promoter, a ribosome binding site for translation initiation, a transcription terminator, and other regulatory elements. The promoter may be a promoter suitable for expression in a production cell.

The present invention also provides a host cell comprising a nucleotide molecule encoding the antigen binding polypeptide of the present invention. The host cells can be used for the production of the antigen binding polypeptide. The host cell can be transiently or non-transiently transfected with the expression vector of the present invention. In a preferred embodiment, the cell is selected from CHO or its derivative cell lines, or 293T or its derivative cell lines.

In some embodiments, the antigen binding polypeptide of the present invention is a single domain antibody (VHH) or a heavy chain antibody. In some embodiments, the antigen binding polypeptide is a humanized antibody and/or a fully humanized antibody. In some embodiments, the antigen binding polypeptide is an antibody fragment, such as Fv, Fab, Fab′, scFv, diabody or F(ab′)₂. In some embodiments, the antigen binding polypeptide is a multispecific antibody, such as a bispecific antibody or a trispecific antibody. In some embodiments, the antigen binding polypeptide of the present invention is a VHH antibody. In some embodiments, the antigen binding polypeptide of the present invention is a VHH-Fc antibody formed by fusion of VHH to Fc from human immunoglobulin, in which the human immunoglobulin is preferably IgG, and further preferably IgG1.

The present invention further provides a fusion protein comprising the antigen binding polypeptide of the present invention. In an embodiment, the fusion protein is a chimeric antigen receptor (CAR), which comprises an extracellular domain, a transmembrane domain and an intracellular signal transduction domain. The extracellular domain comprises the antigen binding polypeptide of the present invention, such as a nanobody of the present invention.

In some embodiments, the transmembrane domain of the CAR may be a transmembrane domain commonly used in the art, such as a transmembrane domain from T cell costimulatory molecules.

In some embodiments, the intracellular signal transduction domain of the CAR may comprise an immunoreceptor tyrosine-based activation motif (ITAM). In some embodiments, the intracellular signal transduction domain may comprise an immunoreceptor tyrosine-based inhibition motif (ITIM). In some embodiments, the intracellular signal transduction domain may comprise an intracellular domain of a molecule selected from the group consisting of Fcγ receptor (FcγR), Fcε receptor (FcεR), Fcα receptor (FcαR), neonatal Fc receptor (FcRn), CD3, CD3ζ, CD3γ, CD3δ, CD3ε, CD4, CD5, CD8, CD21, CD22, CD28, CD32, CD40L (CD154), CD45, CD66d, CD79a, CD79b, CD80, CD86, CD278 (also known as ICOS), CD247ζ, CD247η, DAP10, DAP12, FYN, LAT, Lck, MAPK, MHC complex, NFAT, NF-κB, PLC-γ, iC3b, C3dg, C3d and Zap70.

In some embodiments, the CAR may also comprise a costimulatory domain. In some embodiments, the costimulatory domain may comprise the following signal transduction domains: MHC class I molecule, TNF receptor protein, immunoglobulin-like protein, cytokine receptor, integrin, signaling lymphocyte activation molecule (SLAM protein), NK cell activation receptor or Toll ligand receptor.

### III. Use

The antigen binding polypeptide or antibody, such as nanobody, of the present invention, can specifically bind BAFFR with high affinity, thereby inhibiting BAFF-BAFFR interaction. Therefore, it is particularly suitable for use in the treatment of autoimmune diseases and tumors.

In an embodiment, the antigen binding polypeptide or antibody, such as nanobody, of the present invention, is used to treat subjects suffering from autoimmune diseases. For example, the subjects suffering from autoimmune diseases have an increased expression level of BAFF compared with healthy subjects.

In an embodiment, the antigen binding polypeptide or antibody, such as nanobody, of the present invention, is used to treat subjects suffering from tumors, especially malignant tumors.

In the context of the present invention, the subject is a vertebrate, preferably a mammal, such as a human. The mammal includes, but is not limited to, rats, apes, humans, farm animals, sports animals and pets. Subjects in a broad sense also include tissues, cells and their progenies of biological entities obtained in vivo or cultured in vitro.

The antigen binding polypeptide, antibody, nanobody or fusion protein of the present invention may be comprised in a pharmaceutical composition. The pharmaceutical composition can be used for preventive and/or therapeutic treatment.

In therapeutic use, the pharmaceutical composition of the present invention can be given to a subject having a disease in an amount sufficient to cure or at least partially prevent progression of the symptoms of the disease. The amount effective for treatment can vary according to the severity of the disease, the course of the disease, the previous treatment, the health status, the weight, and the reaction to the drug of the subject, and the judgment of the physician. The antigen binding polypeptide, antibody, nanobody or fusion protein of the present invention can be combined with other therapeutic agents, such as for sequential administration or simultaneous administration.

In preventive use, the pharmaceutical composition of the present invention can be administered before, during or after the occurrence of a disease. For example, the pharmaceutical composition of the present invention can be used as a preventive agent to prevent the occurrence of a disease.

The antigen binding polypeptide, antibody, nanobody, fusion protein or pharmaceutical composition of the present invention can be administered by injection, such as direct injection, stereotactic injection, and injection through a micropump infusion system. The injection can be carried out through intravenous, parenteral, intraperitoneal and/or subcutaneous routes, so as to deliver the antigen binding polypeptide, antibody, nanobody, fusion protein or pharmaceutical composition to the cells, tissues or organs of the subject.

The present invention is further described below in conjunction with specific examples. It is to be understood that these examples are merely illustrative of the present invention and are not intended to limit the scope of the present invention. For experimental methods where no specific conditions are given in the following examples, conventional conditions or conditions recommended by the manufacturer are followed. The reagent for which no specific source is not indicated is a commercially available conventional reagent.

### EXAMPLES

The present invention will be further described below in connection with specific examples. However, it is worth noting that the following examples are intended to illustrate the present invention, instead of limiting the present invention; and technical solutions similar or equivalent to those in the present invention are all embraced in the scope of protection of the present invention. Where specific techniques or conditions are not specified in the examples, the operation is carried out following the conventional technical methods and instrument instructions in the art. The reagents or instruments for which no manufacturers are noted are all common products commercially available from the market.

### Example 1. Production of Anti-BAFFR Nanobodies by Phage Display

In this example, a variety of antibody coding fragments were obtained by immunizing alpaca with human BAFFR as an antigen, and then a candidate antibody clone was screened by a phage library and a mammalian expression system. Specifically, specific steps of the screening process of the anti-BAFFR nanobodies were as follows.

a. Preparation of Antigen

According to the amino acid sequence and nucleotide sequence of human BAFFR, the antigen that can effectively induce alpacas to produce specific antibodies against human BAFFR was analyzed and designed. Human IgG1 Fc was linked to the C end, to obtain a modified antigen, which was designated as “human BAFFR hFC antigen”. The amino acid sequence of the antigen is as shown in SEQ ID NO: 33, and the coding sequence therefor is as shown in SEQ ID NO: 32.

b. Immunization of Alpaca

The human BAFFR hFC antigen obtained in Step a was completely and evenly mixed with an equal volume of Freund's adjuvant, and subcutaneously injected into alpaca.

Specifically, on day 1, alpacas were immunized with an emulsified mixture of 500 μg of human BAFFR hFC antigen with an equal volume of Freund's complete adjuvant, and four times of booster immunization were carried out with an emulsified mixture of 250 μg of human BAFFR hFC antigen with an equal volume of Freund's complete adjuvant on days 21, 42, and 63, and 84 respectively. 7 days after the first 4 immunizations, 10 ml of peripheral blood were collected from alpacas and the anti-BAFFR serum titer in the blood was detected by ELISA.

The specific steps of ELISA detection were as follows: Human BAFFR his antigen was diluted to 2 μg/mL with a 0.05 M carbonate buffer (pH=9.6) and added to a plate in an amount of 100 μL/well. The plate was coated overnight at 4° C. The coating solution was discarded and the plate was washed 3 times with PBST. 300 μL of 5% skim milk was added to each well and the plate was blocked at 37° C. for 1 h. The plate was washed 3 times with a PBST buffer. 100 μL/well of a serum diluent (fold-dilution from 1:2000) was added and incubated at 37° C. for 45 min. The plate was washed 5 times with PBST. 100 μL goat anti-alpaca IgG (H+L) HRP (Chengdu NBbiolab Biotechnology Co., Ltd., Cat #: S001H, Chengdu NBbiolab Biotechnology Co., Ltd., 1:1W dilution with PBS) was added to each well, and incubated at 37° C. for 45 min. The plate was washed 5 times with PBST. ATMB developing solution (100 μL/well) was added and incubated at 37° C. for 5 min. The reaction was terminated by adding a termination solution (50 μL/well), and the optical density was measured at 450 nm.

1 week after the 5th immunization, 50 mL of peripheral blood was collected from alpaca and mononuclear cells were isolated.

c. Library Construction

RNA of PBMCs obtained in Step b was extracted, and a target gene fragment was obtained by nested PCR after reverse transcription. The target gene fragment was cloned into a eukaryotic expression vector, and the obtained expression vector was transformed into competent cells to construct a BAFFR-VHH phage display library. The specific steps were as follows.

PBMC cells screened in Step b were used as a template, total RNA was extracted from the cells by using RNAiso Plus, and the RNA was reverse transcribed into cDNA by using PrimeScript™ II 1st Strand cDNA Synthesis Kit. The target fragment was obtained by nested PCR. The amplification system for the target fragment was shown in Table 1 below, and the amplification procedure was shown in Table 2 below.

TABLE 1
First-round reaction system of nested PCR

| Reagent | Volume (μL) |
|---|---|
| cDNA template (5-fold dilution) | 5 |
| Upstream primer: NPR-19001 (5′ CTTGGTGGTCCTGGCTGC 3′; | 1.5/1.5 |
| SEQ ID NO: 34) | |
| Downstream primer: NPR-19002 (5′ GGTACGTGCTGTTGAACTGTTCC | |
| 3′; SEQ ID NO: 35) | |
| dNTP Mixture | 4 |
| 10×PrimeSTAR Buffer (Mg2+ plus) | 5 |
| PrimeSTAR ® HS DNA Polymerase | 0.5 |
| ddH₂O | q.s. to 50 |
| ddH₂O | q.s. to 50 |

TABLE 2
Second-round reaction system of nested PCR

| Reagent | Volume (μL) |
|---|---|
| cDNA template (5-fold dilution) | 5 |
| Upstream primer: NPR-19003 (SEQ ID NO: 36) | 1.5/1.5 |
| 5′-CATGCCATGACTGTGGCCCAGGCGGCCCAGKTGCAGCTCGTGG | |
| AGTC-3′ | |
| Downstream primer: NPR-19004 (SEQ ID NO: 37) | |
| 5′-CATGCCATGACTGTGGCCCAGGCGGCCCAGKTGCAGCTCGTGG | |
| AGTC-3′ or | |
| NPR-19005 (SEQ ID No: 38) | |
| 5′-CATGCCATGACTCGCGGCCGGCCTGGCCGCTGGGGTCTTCGCT | |
| GTGGTGCG-3′ | |
| dNTP Mixture | 4 |
| 10×PrimeSTAR Buffer (Mg2+ plus) | 5 |
| PrimeSTAR ® HS DNA Polymerase | 0.5 |
| ddH₂O | q.s. to 50 |

The amplified nucleotide fragment encoding alpaca VHH was cloned into the eukaryotic expression vector pComb3XSS (Chengdu NBbiolab Biotechnology Co., Ltd.). The produced recombinant vector was transformed into TG1 competent cells by electric shock, to obtain a VHH phage display library. To further identify whether the BAFFR-VHH phage display library was successfully constructed, the library was cultured on 2-YT-A plate, and 48 clones were picked up from the formed colonies for sequencing.

The sequencing results shows that the successful insertion rate of the clone is 100%. According to the number of library transformants, the library insertion rate and the diversity sequencing results, the library capacity is calculated to be 1.92×10⁹, showing that the library capacity and diversity of the obtained BAFFR-VHH phage display library are good.

d. Nanobody Production

The phage display library obtained in Step c was used for panning and positive clones were screened. The coding sequence of VHH antibody screened was fused with the coding sequence of Human IgG1 Fc, constructed into the expression vector of pTT5 (Chengdu NBbiolab Biotechnology Co., Ltd.) and transfected into eukaryotic cells, followed by expression and purification. Finally, anti-human BAFFR nanobody was obtained. The specific method was as follows.

SA-magnetic beads (Suzhou Nanowin Technology Co., Ltd., MPHTSA-300) were taken, and washed 2 times with PBS. Bio-Human BAFFR-his (Acro, BAR-H82E3) antigen was diluted with PBS to a final concentration of 5 μg/ml, and 2×10¹¹ phage library was added, and incubated at 37° C. for 1 h. The incubated mixture was added to a magnetic bead-conjugated tube and shaken at 4° C. for 45 min. The supernatant was removed under the action of a magnetic frame, and the sections were washed 3 times with PBST, then 2 times with PBS. 800 μL of Gly-HCl eluent was added, and incubated at 37° C. for 8 min, to elute the specifically bound phage. The eluate was transferred to a 1.5 mL sterile centrifuge tube and quickly neutralized with 160 μL of a Tris-HCl neutralization buffer. 10 μL was diluted over gradients, the titer was determined, and the panning recovery rate was calculated. The remaining eluates were mixed, amplified, and purified, for the next round of affinity panning.

After two rounds of screening, the supernatant of the monoclonal phage was identified by ELISA after the second round of screening. Bio-Human BAFFR-his antigen was immobilized on a 96-well microplate according to a specification of 2 μg/ml*100 μl, and incubated at 4° C. overnight. The coating solution was discarded and the plate was washed 3 times with PBST. 300 μL of 5% skim milk was added to each well and the plate was blocked at 37° C. for 1 h. The plate was washed 3 times with a PBST buffer, Two-fold dilutions of the phage supernatant were added in an amount of 100 μL/well, and incubated at 37° C. for 45 min. The plate was washed 5 times with PBST. Mouse anti-M13 antibody HRP (Chengdu NBbiolab Biotechnology Co., Ltd.) was added to each well in an amount of 100 μL/well and incubated at 37° C. for 45 min. The plate was washed 5 times with PBST. ATMB developing solution (100 μL/well) was added and incubated at 37° C. for 5 min.

The reaction was terminated by adding a 1 M HCl termination solution in an amount of 50 μL/well, and the optical density was detected at a wavelength of 450 nm. The positive clones (OD450>1) were screened according to the detection results. Human IgG1 Fc was fused to the positive clone and constructed into pTT5 plasmid. The corresponding plasmid was transfected into mammalian cell HEK293T, and expressed on a shaker with 5% carbon dioxide at 37° C. for 7 days. The cell supernatant was collected after culture, and 4 target antibodies were isolated and purified by Protein A affinity packing (Suzhou Nanowin Technology Co., Ltd., 17010-050100), which were designated as NB467-55, NB467-46, NB467-35, and NB467-8.

The VHH amino acid sequences and nucleotide sequences of nanobodies NB467-55, NB467-46, NB467-35, and NB467-8 are shown below.

TABLE 3
Sequence information of NB467-55,
NB467-46, NB467-35, and NB467-8

| Antibody name | Amino acid sequence NO. | Nucleotide sequence NO. |
|---|---|---|
| NB467-55 | 1 | 28 |
| NB467-46 | 2 | 29 |
| NB467-35 | 3 | 30 |
| NB467-8 | 4 | 31 |

### Example 2: Binding of Anti-BAFFR Nanobody to 293T Cells Overexpressing BAFFR

The binding affinity of the 4 anti-BAFFR nanobodies NB467-55, NB467-46, NB467-35, and NB467-8 obtained in Example 1 were characterized by flow cytometry. Moreover, Lanalumab (Chengdu NBbiolab Biotechnology Co., Ltd.), an anti-BAFFR antibody, was used as a positive control antibody.

Specifically, HEK-293T-BAFFR cells were collected from the cell culture flask, washed twice with a FACS buffer and centrifuged. The cells were resuspended in a suitable volume of staining solution for flow cytometry, so that the final cell concentration per tube reached 2×10⁵ cells/mL.

The antibodies NB467-55, NB467-46, NB467-35, and NB467-8 produced in Example 1, and Lanalumab were respectively diluted to 200 nM. 50 μl of the antibody solution was added into a test tube with HEK-293T-BAFFR cells, mixed uniformly, and incubated at 4° C. for 60 min. After incubation, the cells were washed once with a FACS buffer.

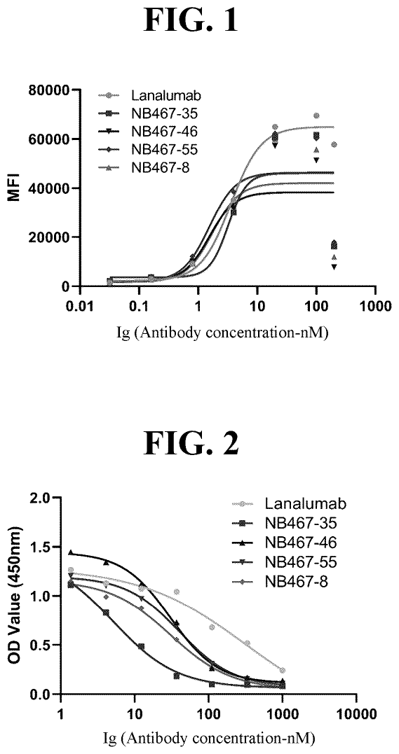

The anti-human FC-647 secondary antibody (Jackson, 109-605-003) was diluted according to the instructions, and 100 μl of the diluted secondary antibody was added to a sample tube. After uniform mixing, the system was incubated at 4° C. for 60 min. After incubation, the cells were washed 3 times with FACS, and the cell precipitate obtained by centrifugation was resuspended in 200 μl of PBS and detected by flow cytometry (Sony, SA3800). The detection results are shown in Table 4 and FIG. 1.

TABLE 4
Binding of nanobody to 293T cells overexpressing BAFF (EC₅₀)

| — | Lanalumab | NB467-35 | NB467-46 | NB467-55 | NB467-8 |
|---|---|---|---|---|---|
| EC₅₀ (nM) | 3.585 | 3.24 | 1.546 | 1.546 | 1.764 |

FIG. 1 shows the average fluorescence intensity (MFI) value of each antibody. Compared with Lanalumab, the nanobodies of the present invention have stronger binding ability to 293T cells that overexpress human BAFFR. Table 4 shows that the EC₅₀ values of the nanobodies of the present invention are all better than or close to that of the control antibody Lanalumab, where NB467-46, NB467-55, and NB467-8 is superior to NB467-35.

### Example 3. Competitive Blocking Ability of Anti-Human BAFFR Nanobodies on BAFF-BAFFR Interaction

In this example, the competitive blocking ability of the 4 anti-BAFFR nanobodies obtained in Example 1 on BAFF-BAFFR interaction was determined. Moreover, Lanalumab (the same as above) was used as a positive control antibody.

Specifically, human BAFFR-his protein (Acro, BAR-HP2H6) was added to a 96-well microplate at 200 ng/well and incubated at 4° C. overnight. On the following day, the plate was washed 3 times with PBST. Then the plate was blocked with 5% w/v skim milk in PBST at 37° C. for 1 h, and then washed with PBS.

The antibody to be tested was 1:3 diluted over gradients from a concentration of 800 nM (diluted with 5% skim milk). Biotin Human BAFF his protein (Acro, BAF-H82Q2) was diluted to 0.2 μg/mL. Control group: 50 μL of diluted Biotin Human BAFF his protein and 50 μL of 5% milk. Experimental group: 50 μL of diluted Biotin Human BAFF his protein and 50 μL of antibody diluted over gradients.

Then, the antibody/Biotin Human BAFF his mixture was added to a plate coated with Human BAFFR-his protein, and incubated at 37° C. for 1 h. The plate was washed 5 times with PBST. The secondary antibody (Chengdu NBbiolab Biotechnology Co., Ltd.) 1:10K diluted with a blocking solution was added and incubate at 37° C. for 1 h. The plate incubated with the secondary antibody was washed 5 times with PBST. 100 μl of a TMB monocomponent developing solution was added to each well, and incubated at 37° C. for 7 min. The reaction was terminated with 50 μL/well of 1 M HCL, and then the absorption value at 450 nm was determined. The median inhibition concentration (IC50 value) was obtained by analyzing the data with Graphpad Prism software. The specific results are shown in Table 5 and FIG. 2.

TABLE 5
Competitive blocking of BAFF-BAFFR interaction by nanobodies

| — | Lanalumab | NB467-35 | NB467-46 | NB467-55 | NB467-8 |
|---|---|---|---|---|---|
| IC₅₀ (nM) | 310.2 | 5.134 | 30.47 | 40.47 | 30.48 |

The results show that compared with Lanalumab, the IC50 values of the nanobodies of the present invention are lower, and the IC50 values are about one tenth or even less of that of Lanalumab, indicating that the competitive blocking ability of the nanobodies of the present invention on BAFF-BAFFR interaction is significantly stronger than that of Lanalumab. FIG. 2 shows the absorption value at 450 nm, indicating that the antibody of the present invention can block the human BAFF-BAFFR interaction, and compared with the reference antibody, the antibody of the present invention has better BAFF-BAFFR blocking activity.

### Example 4. ADCC Activity of Anti-Human BAFFR Nanobodies

In this example, the ADCC activity of the 4 anti-BAFFR nanobodies obtained in Example 1 was determined in this example. Moreover, Lanalumab (the same as above) was used as a positive control antibody.

Specifically, HEK293T-BAFFR cells as in Example 2 were digested, collected by centrifugation, and resuspended in DEME+1% FBS. 35000 cells were plated in each well in 96-well assay plate in a volume of 50 μl. Jurkat-NFAT-CD16a cells (purchased from Sichuan Swiftbio Technology Co., Ltd.) were collected by centrifugation, and resuspended in RPMI 1640+1% FBS. 100000 cells were plated in each well in a volume of 50 μl.

The antibody to be tested was diluted with RPMI1640+1% FBS. The antibody was 5-fold diluted starting from a concentration of 50 g/mL, and a total of 11 concentration points were set. The diluted antibody was added to the plate at 100 μl/well, and 50 μg/mL isotype control was added as a negative control. The plate was incubated in an incubator with carbon dioxide at 37° C. for 5 h. After incubation, 30u 1 of bio-lite luciferase assay system luciferase substrate was added to each well, incubated for 3 min with shaking, and detected on SpectraMax i3x.

The EC₅₀ value was obtained by analyzing the data with Graphpad Prism software. The test results are shown in Table 6.

TABLE 6
EC₅₀ values of four antibodies and the control antibody

| Antibody | Lanalumab | NB467-35 | NB467-46 | NB467-55 | NB467-8 |
|---|---|---|---|---|---|
| Slope | 1.210 | 0.6831 | 0.9969 | 1.081 | 0.6987 |
| (Hill- | | | | | |
| Slope) | | | | | |
| EC₅₀ | 56.47 | 2.668 | 4.555 | 5.092 | 2.890 |
| (ng/ml) | | | | | |

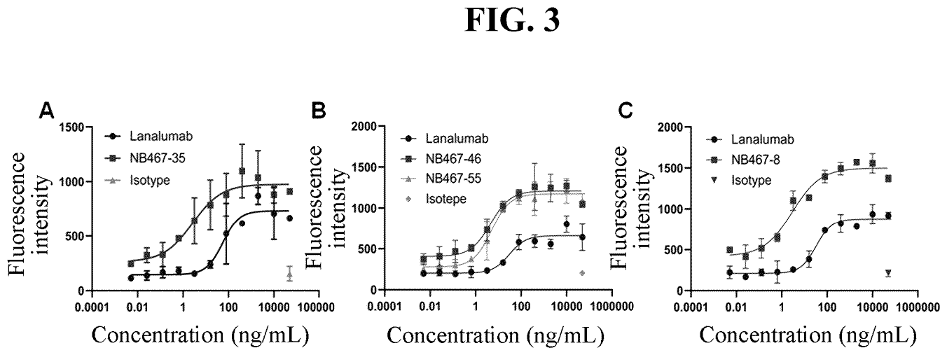

The results in FIG. 6 shows that the nanobodies of the present invention can induce ADCC effect with an EC₅₀ significantly lower than that of the reference antibody, showing a stronger ADCC effect than that of the reference antibody Lanalumab. The fluorescence intensity-concentration curve of FIG. 3 shows that the antibody of the present invention shows stronger fluorescence intensity than that of the reference antibody Lanalumab, indicating a higher ADCC effect. Isotype stands for the isotype control antibody.

Specific embodiments of the present invention have been described above, which are provided for understanding and implementing the present invention by those skilled in the art. Many modifications to these embodiments will be obvious to those skilled in the art, and the general principles defined herein can be implemented in other embodiments without departing from the scope of the present invention. Therefore, the present invention is not limited to the embodiments shown herein, but falls within the widest scope consistent with the principles and novel features disclosed herein

TABLE 7
Sequence information

| | | | SEQ |
|---|---|---|---|
| Name | Type | Sequence | ID NO: |
| NB467-55 | AA | EVQVVESGGDLVQPGGSLKLSCQASGFSLTYYAIGWFR | 1 |
| | | QAPGKEREEISCISGDGGMTYYADPVKGRFTISRDNGRN | |
| | | TVYLQMNSLKPEDTAVYYCAADVYYNGNYYSRSCNPAE | |
| | | SKYWGQGTQVTVSA | |
| NB467-46 | AA | EVQVVESGGGLVQPGGSLRLSCAASGFSLTYYAIGWFR | 2 |
| | | QAPGKEREGVACISGGGDQLYYTDSVKGRFTISRDNAKN | |
| | | TVYLHMNSLKPEDTAVYYCAADVYYSGSYNYRSCNPAES | |
| | | KYWGQGTQVTVSS | |
| NB467-35 | AA | QVQLVESGGGLVQPGGSLRLSCAASGFSLEYYAIGWFR | 3 |
| | | QTPGKEREAISCIDSPAKSTYYADSVKGRFTISRDNSKNT | |
| | | VYLQMNSLKAEDTAVYYCAADVYYRGSYLYRSCNPTESK | |
| | | YWGQGTQVTVSS | |
| NB467-8 | AA | QVQLVESGGGLVQPGGSLRLSCAASGFSLEYYAIGWFR | 4 |
| | | HIPGKEREAVSCISSPGDSTWYVDSVKGRFTISRDNAKNT | |
| | | VYLQMNSLKPEDTAVYHCAADVYYSGSYLSRSCNPHESK | |
| | | YWGQGTQVTVSS | |
| NB467-55- | AA | EVQVVESGGDLVQPGGSLKLSCQAS | 5 |
| FR1 | | | |
| NB467-46- | AA | EVQVVESGGGLVQPGGSLRLSCAAS | 6 |
| FR1 | | | |
| NB467-35- | AA | QVQLVESGGGLVQPGGSLRLSCAAS | 7 |
| FR1 | | | |
| NB467-6- | AA | QVQLVESGGGLVQPGGSLRLSCAAS | 7 |
| FR1 | | | |
| NB467-55- | AA | GFSLTYYA | 8 |
| CDR1 | | | |
| NB467-46- | AA | GFSLTYYA | 8 |
| CDR1 | | | |
| NB467-35- | AA | GFSLEYYA | 9 |
| CDR1 | | | |
| NB467-8- | AA | GFSLEYYA | 9 |
| CDR1 | | | |
| NB467-55- | AA | IGWFRQAPGKEREEISC | 10 |
| FR2 | | | |
| NB467-46- | AA | IGWFRQAPGKEREGVAC | 11 |
| FR2 | | | |
| NB467-35- | AA | IGWFRQTPGKEREAISC | 12 |
| FR2 | | | |
| NB467-8- | AA | IGWFRHIPGKEREAVSC | 13 |
| FR2 | | | |
| NB467-55- | AA | ISGDGGMT | 14 |
| CDR2 | | | |
| NB467-46- | AA | ISGGGDQL | 15 |
| CDR2 | | | |
| NB467-35- | AA | IDSPAKST | 16 |
| CDR2 | | | |
| NB467-6- | AA | ISSPGDST | 17 |
| CDR2 | | | |
| NB467-55- | AA | YYADPVKGRFTISRDNGRNTVYLQMNSLKPEDTAVYYC | 18 |
| FR3 | | | |
| NB467-46- | AA | YYTDSVKGRFTISRDNAKNTVYLHMNSLKPEDTAVYYC | 19 |
| FR3 | | | |
| NB467-35- | AA | YYADSVKGRFTISRDNSKNTVYLQMNSLKAEDTAVYYC | 20 |
| FR3 | | | |
| NB467-8- | AA | WYVDSVKGRFTISRDNAKNTVYLQMNSLKPEDTAVYHC | 21 |
| FR3 | | | |
| NB467-55- | AA | AADVYYNGNYYSRSCNPAESKY | 22 |
| CDR3 | | | |
| NB467-46- | AA | AADVYYSGSYNYRSCNPAESKY | 23 |
| CDR3 | | | |
| NB467-35- | AA | AADVYYRGSYLYRSCNPTESKY | 24 |
| CDR3 | | | |
| NB467-8- | AA | AADVYYSGSYLSRSCNPHESKY | 25 |
| CDR3 | | | |
| NB467-55- | AA | WGQGTQVTVSA | 26 |
| FR4 | | | |
| NB467-46- | AA | WGQGTQVTVSS | 27 |
| FR4 | | | |
| NB467-35- | AA | WGQGTQVTVSS | 27 |
| FR4 | | | |
| NB467-6- | AA | WGQGTQVTVSS | 27 |
| FR4 | | | |
| NB467-55 | DNA | GAGGTGCAGGTGGTGGAGTCTGGGGGAGACTTGGTG | 28 |
| | | CAGCCTGGGGGGTCTCTGAAACTCTCCTGTCAAGCCT | |
| | | CTGGATTCAGTTTGACGTATTATGCCATAGGCTGGTTC | |
| | | CGCCAGGCCCCAGGAAAGGAACGTGAGGAAATCTCAT | |
| | | GTATTAGTGGTGATGGTGGTATGACATACTATGCAGAC | |
| | | CCCGTGAAGGGCCGATTCACCATCTCCAGAGACAACG | |
| | | GCAGGAACACGGTGTATCTGCAAATGAACAGCCTGAAA | |
| | | CCTGAGGACACAGCCGTTTATTACTGTGCAGCAGACGT | |
| | | ATACTATAATGGCAATTACTACAGCCGCTCATGTAACC | |
| | | CTGCTGAGTCAAAGTACTGGGGCCAGGGGACCCAGGT | |
| | | CACCGTCTCCGCA | |
| NB467-46 | DNA | GAGGTGCAGGTGGTGGAGTCTGGGGGAGGCTTGGTG | 29 |
| | | CAACCTGGGGGGTCTCTGAGACTCTCCTGTGCAGCCT | |
| | | CTGGATTCAGTTTGACTTATTATGCCATAGGCTGGTTC | |
| | | CGCCAGGCCCCAGGGAAGGAGCGTGAGGGGGTTGCA | |
| | | TGTATTAGTGGTGGGGGTGACCAGCTTTATTATACAGA | |
| | | CTCCGTGAAGGGCCGATTCACCATCTCCAGAGACAAT | |
| | | GCCAAGAACACGGTGTATCTGCACATGAACAGCCTGAA | |
| | | ACCTGAGGACACAGCCGTTTATTACTGTGCAGCAGATG | |
| | | TCTACTATAGTGGTAGTTACAACTACCGGTCATGTAAC | |
| | | CCTGCTGAGTCAAAATACTGGGGCCAGGGGACCCAGG | |
| | | TCACCGTGTCCTCA | |
| NB467-35 | DNA | CAGGTGCAGCTCGTGGAGTCCGGGGGAGGCTTGGTG | 30 |
| | | CAGCCTGGGGGGTCTCTGAGACTCTCCTGTGCAGCCT | |
| | | CTGGATTCAGTTTGGAGTATTATGCCATAGGCTGGTTC | |
| | | CGCCAGACCCCAGGAAAGGAGCGTGAGGCGATCTCAT | |
| | | GTATTGATAGTCCTGCTAAGAGCACGTACTATGCAGAC | |
| | | TCCGTGAAGGGCCGATTCACCATCTCCAGAGACAACTC | |
| | | CAAGAACACGGTGTATCTGCAAATGAACAGCCTGAAAG | |
| | | CTGAGGACACAGCCGTTTATTACTGTGCAGCAGATGTA | |
| | | TACTATCGCGGTAGTTACTTGTACCGCTCATGTAACCC | |
| | | TACTGAGTCAAAATACTGGGGCCAGGGGACCCAGGTC | |
| | | ACCGTCTCCTCA | |
| NB467-8 | ONA | CAGGTGCAGCTCGTGGAGTCGGGGGGAGGCTTGGTG | 31 |
| | | CAGCCTGGGGGGTCTCTGAGACTCTCCTGTGCAGCGT | |
| | | CTGGATTCAGTTTGGAGTATTATGCCATAGGCTGGTTC | |
| | | CGCCACATCCCAGGGAAAGAGCGTGAGGCGGTCTCAT | |
| | | GTATTAGTAGTCCTGGTGATAGTACGTGGTATGTAGAC | |
| | | TCCGTGAAGGGCCGATTCACCATCTCCAGAGACAACG | |
| | | CCAAGAACACGGTGTATCTGCAAATGAACAGCCTGAAA | |
| | | CCTGAGGACACAGCCGTTTATCACTGTGCAGCAGACG | |
| | | TATACTATAGTGGTAGTTACCTGAGCCGCTCATGTAAC | |
| | | CCGCATGAATCAAAATACTGGGGCCAGGGGACCCAGG | |
| | | TCACCGTCTCCTCA | |
| Coding | ONA | ATGGGCTGGTCCTGCATCATCCTGTTTCTGGTC | 32 |
| sequence | | GCTACCGCCACAGGCGTCCACTCCTCCCTGAG | |
| of human | | GGGCAGAGACGCCCCCGCTCCAACACCTTGCG | |
| BAFFR | | TGCCTGCCGAGTGCTTCGATCTGCTGGTGAGG | |
| hFC | | CACTGCGTGGCCTGCGGCCTGCTGAGGACCCC | |
| antigen | | TAGGCCTAAGCCCGCCGGCGCTAGCAGCCCAG | |
| | | CTCCTAGAACAGCCCTGCAGCCCCAGGAGAGC | |
| | | GTGGGCGCTGGAGCTGGAGAGGCCGCTCTGCC | |
| | | CCTGCCTGGCCTGGAACCAAAATCTTGTGACA | |
| | | AAACCCACACATGCCCACCTTGTCCCGCCCCT | |
| | | GAACTGCTGGGCGGACCTTCTGTCTTTCTGTTC | |
| | | CCCCCCAAACCCAAGGATACACTGATGATCTC | |
| | | TAGAACCCCCGAGGTCACATGTGTCGTCGTGG | |
| | | ATGTGTCCCATGAGGACCCTGAAGTGAAATTC | |
| | | AACTGGTACGTGGACGGAGTGGAAGTCCATA | |
| | | ACGCCAAAACCAAACCACGAGAGGAACAGTA | |
| | | CAATAGCACATATCGAGTCGTGTGTGTGCTGA | |
| | | CTGTGCTGCATCAGGATTGGCTGAACGGCAAG | |
| | | GAATACAAATGTAAAGTGTCCAATAAGGCACT | |
| | | GCCCGCTCCTATTGAAAAAACAATTTCAAAGG | |
| | | CAAAAGGCCAGCCTCGTGAACCTCAGGTGTAC | |
| | | ACACTGCCACCTTCTCGGGAGGAAATGACCAA | |
| | | AAACCAGGTGTCACTGACCTGTCTGGTCAAGG | |
| | | GCTTTTACCCTTCCGATATTGCTGTCGAGTGGG | |
| | | AGAGTAACGGCCAGCCCGAAAACAACTACAA | |
| | | AACCACCCCTCCTGTGCTGGATTCCGATGGCT | |
| | | CATTCTTCCTGTACTCTAAACTGACCGTGGATA | |
| | | AGAGTCGCTGGCAGCAGGGCAATGTGTTTTCT | |
| | | TGCTCCGTGATGCATGAGGCACTGCACAACCA | |
| | | CTACACCCAGAAATCCCTGTCACTGTCTCCCG | |
| | | GAAAATAA | |
| Amino | | MGWSCIILFLVATATGVHSSLRGRDAPAPTPCVP | 33 |
| acid | | AECFDLLVRHCVACGLLRTPRPKPAGASSPAPRT | |
| sequence | | ALQPQESVGAGAGEAALPLPGLEPKSCDKTHTC | |
| of human | | PPCPAPELLGGPSVFLFPPKPKDTLMISRTPEVTC | |
| BAFFR | | VVVDVSHEDPEVKFNWYVDGVEVHNAKTKPRE | |
| hFC | | EQYNSTYRVVSVLTVLHQDWLNGKEYKCKVSN | |
| antigen | | KALPAPIEKTISKAKGQPREPQVYTLPPSREEMT | |
| | | KNQVSLTCLVKGFYPSDIAVEWESNGQPENNYK | |
| | | TTPPVLDSDGSFFLYSKLTVDKSRWQQGNVFSC | |
| | | SVMHEALHNHYTQKSLSLSPGK | |
| Nucleotide | DNA | ATGAGGCGAGGGCCCCGGAGCCTGCGGGGCA | 33 |
| sequence | | GGGACGCGCCAGCCCCCACGCCCTGCGTCCCG | |
| of human | | GCCGAGTGCTTCGACCTGCTGGTCCGCCACTG | |
| BAFFR | | CGTGGCCTGCGGGCTCCTGCGCACGCCGCGGC | |
| | | CGAAACCGGCCGGGGCCAGCAGCCCTGCGCC | |
| | | CAGGACGGCGCTGCAGCCGCAGGAGTCGGTG | |
| | | GGCGCGGGGGCCGGCGAGGGGGGGGTGCCCC | |
| | | TGCCCGGGCTGCTCTTTGGCGCCCCCGCGCTG | |
| | | CIGGGCCTGGCACTGGTCCTGGCGCTGGTCCT | |
| | | GGTGGGTCTGGTGAGCTGGAGGCGGCGACAG | |
| | | CGGCGGCTTCGCGGCGCGTCCTCCGCAGAGGC | |
| | | CCCCGACGGAGACAAGGACGCCCCAGAGCCC | |
| | | CTGGAGAAGGTCATCATTGTGTCTCCGGGAAT | |
| | | CTCTGATGCCACAGCTCCTGCCTGGCCTCCTCC | |
| | | TGGGGAAGACCCAGGAACCACCCCACCTGGC | |
| | | CACAGTGTCCCTGTCCCAGCCACAGAGCTGGG | |
| | | CTCCACTGAACTGGTGACCACCAAGACGGCCG | |
| | | GCCCTGAGCAACAATAG | |
| Amino | AA | MRRGPRSLRGRDAPAPTPCVPAECFDLLVRHCV | 40 |
| acid | | ACGLLRTPRPKPAGASSPAPRTALQPQESVGAG | |
| sequence | | AGEAALPLPGLLFGAPALLGLALVLALVLVGLV | |
| of human | | SWRRRQRRLRGASSAEAPDGDKDAPEPLDKVIIL | |
| BAFFR | | SPGISDATAPAWPPPGEDPGTTPPGHSVPVPATE | |
| | | LGSTELVTTKTAGPEQQ | |

What is claimed is:

1. An antigen binding polypeptide specifically binding B lymphocyte stimulator factor receptor BAFFR, comprising one group of CDR1, CDR2 and CDR3 selected from
(a) the CDR1 having an amino acid sequence as shown in SEQ ID NO: 8;
the CDR2 having an amino acid sequence as shown in SEQ ID NO: 14; and
the CDR3 having an amino acid sequence as shown in SEQ ID NO: 22;
(b) the CDR1 having an amino acid sequence as shown in SEQ ID NO: 8;
the CDR2 having an amino acid sequence as shown in SEQ ID NO: 15; and
the CDR3 having an amino acid sequence as shown in SEQ ID NO: 23;
(c) the CDR1 having an amino acid sequence as shown in SEQ ID NO: 9;
the CDR2 having an amino acid sequence as shown in SEQ ID NO: 16; and
the CDR3 having an amino acid sequence as shown in SEQ ID NO: 24; or
(d) the CDR1 having an amino acid sequence as shown in SEQ ID NO: 9;
the CDR2 having an amino acid sequence as shown in SEQ ID NO: 17; and
the CDR3 having an amino acid sequence as shown in SEQ ID NO: 25.

2. The antigen binding polypeptide according to claim 1, wherein the antigen binding polypeptide is a VHH single domain antibody, or a VHH-Fc antibody formed by fusion of VHH to Fc derived from human immunoglobulin.

3. The antigen binding polypeptide according to claim 2, comprising an amino acid sequence as shown in any one of SEQ ID NOs: 1-4; or wherein the VHH comprises an amino acid sequence as shown in any one of SEQ ID NOs: 1-4.

4. An isolated nucleic acid molecule, comprising a nucleotide sequence encoding the antigen binding polypeptide according to claim 1.

5. The isolated nucleic acid molecule according to claim 4, wherein the nucleotide sequence comprises or consists of a nucleotide sequence as shown in any one of SEQ ID NOs: 28-31.

6. A recombinant expression vector, comprising the isolated nucleic acid molecule according to claim 4.

7. A host cell, comprising the recombinant expression vector according to claim 6.

8. A pharmaceutical composition, comprising the antigen binding polypeptide according to claim 1 and a pharmaceutically acceptable carrier.

9. Use of the antigen binding polypeptide according to claim 1 in the preparation of drugs for treating tumors or autoimmune diseases.

10. A fusion protein, comprising the antigen binding polypeptide according to claim 1.
